(19) United States
(12) Patent Application Publication
(10) Pub. No.: US 2022/0409436 A1
(43) Pub. Date: Dec. 29, 2022

(54) DRESSING HAVING METAL COATING

(71) Applicant: Aatru Medical, LLC, Cleveland, OH (US)

(72) Inventor: John Buan, Maple Grove, MN (US)

(21) Appl. No.: 17/358,581

(22) Filed: Jun. 25, 2021

(51) Int. Cl. A61F 13/00; A61F 13/02; A61M 1/00

(52) U.S. Cl. CPC A61F 13/00068; A61F 13/0246; A61M 1/90; A61F 13/0289; A61F 2013/00114

(57) ABSTRACT

A dressing includes a drape, a metal coating provided on a skin-facing surface of the drape, a pressure-sensitive adhesive provided on a lower surface of the metal coating, and a gasket having a frame shape. The drape is a thin film. The gasket includes an inner edge defining an opening and an outer edge offset inwardly from an outer periphery of the drape. The gasket is in contact with the metal coating and leaves a margin of pressure-sensitive adhesive around the gasket.

### BACKGROUND

Negative pressure is a term used to describe a pressure that is below normal atmospheric pressure. When used with treating a wound it can be referred to as negative pressure wound therapy (“NPWT”). One type of dressing used with NPWT includes a porous foam positioned on the wound and a dressing cover over the porous foam. A drainage tube connected with a pump siphons exudate from the wound after a water tight seal around the wound has been provided.

Known negative pressure systems typically employ a mechanical pump that draws air from an enclosed volume through the mechanical pump into ambient. Leakage in these systems, which can include transmission of air through the dressing itself, is not as critical because the mechanical pump typically can overcome the effect of a relatively small flow of air entering the enclosed volume by way of leakage in the system. Even though drapes provided with negative pressure dressings are typically designed to maintain negative pressure underneath the drape upon application of a vacuum, advancements can be made with regard to these dressings used with negative pressure to allow the dressing to maintain negative pressure over a longer period of time without having to continually draw a vacuum from beneath the drape. Additionally, these drapes can be improved for use in areas other than negative pressure wound therapy.

### SUMMARY

In view of the foregoing, a dressing includes a drape, a metal coating provided on a skin-facing surface of the drape, a pressure-sensitive adhesive provided on a lower surface of the metal coating, and a gasket having a frame shape. The drape is a thin film. The gasket includes an inner edge defining an opening and an outer edge offset inwardly from an outer periphery of the drape. The gasket is in contact with the adhesive layer on the lower surface of the metal coating and leaves a margin of pressure-sensitive adhesive around the gasket.

For the dressing mentioned above, the drape can have a thickness less than 0.05 mm and the metal coating can have a thickness of less than 4.0×10⁻⁵ mm. Also, the metal coating can have a thickness of less than 2.0×10⁻⁵ mm.

For each dressing mentioned above in any of the preceding paragraphs, the dressing can further include a casting sheet, which is a polymer. The drape is cast onto the casting sheet such that the casting sheet is provided on an outer surface of the drape, which is opposite to the skin-facing surface, when the dressing is finally assembled. The casting sheet can have a frame shape with an internal edge defining a casting sheet opening. An external edge of the casting sheet can be coincident with the outer periphery of the drape.

For each dressing mentioned above in any of the preceding paragraphs, the drape can include an aperture extending through the drape and the metal coating. The aperture is surrounded by the gasket. Such a dressing with the aperture extending through the drape and metal coating can further include a hose fitting or a valve mounted to the drape and in fluid communication with the aperture. These dressings can be used in combination with a vacuum source, where the vacuum source is in fluid communication with the hose fitting or the valve. In addition to being a mechanical or an electro-mechanical pump, the vacuum source can also be a reactor configured to consume at least one gas found in air, such as a solid-state scavenger that reacts with a gas in air, e.g., oxygen, to remove the gas from beneath the drape.

For each dressing mentioned above in any of the preceding paragraphs, the dressing can further include an absorbent pad affixed to the metal coating and the drape via the pressure-sensitive adhesive. For such a dressing, the absorbent pad is surrounded by the gasket.

For each dressing mentioned above in any of the preceding paragraphs, the gasket can include a backing film having a frame shape with an inner border defining a portion of the opening of the gasket and an outer border offset inwardly from the outer periphery of the drape. The backing film is affixed to the metal coating and the drape with the metal coating via the pressure-sensitive adhesive leaving the margin of pressure-sensitive adhesive around the backing film. The gasket can further include a silicone gel provided on the backing film surrounding the opening; however, other types of material, such as a hydrogel, could also be employed with the gasket.

A method of assembling a dressing includes providing as a pre-manufactured roll a casting sheet, a drape and metal coating provided on a skin-facing surface of the drape and coating a pressure-sensitive adhesive on the metal coating. A backing film having a silicone gel deposited thereon is applied onto the pressure-sensitive adhesive to affix the backing film with the silicone to the drape with the metal coating provided thereon. The backing film has frame shape with an inner border defining an opening and an outer border offset from an outer periphery of the drape leaving a margin of pressure-sensitive adhesive around the backing film, and the silicone gel surrounds the opening.

The method described in the aforementioned paragraph can further include forming an aperture through the casting sheet, the drape and the metal coating. The method of assembling the dressing can further include kiss cutting the casting sheet around the aperture, removing an inside portion of casting sheet, and affixing a hose fitting or a valve to the drape that is in fluid communication with the aperture.

The method in any of the above two paragraphs can further include applying an absorbent pad onto the pressure-sensitive adhesive to affix the absorbent pad to the drape with the metal coating provided thereon. The method can further include kiss cutting the casting sheet around the absorbent pad, and removing an inside portion of casting sheet.

### BRIEF DESCRIPTION OF THE DRAWINGS

FIG. 1 is an exploded perspective view of a dressing including a valve.

FIG. 2 is an exploded schematic side view of the dressing having a fitting instead of the valve depicted in FIG. 1, and a vacuum source connected with the fitting.

FIG. 3 a schematic side view of an upper part of the dressing having the valve and another example of a vacuum source for use with the dressing.

FIG. 4 is a plan view of a drape for the dressing showing a metal coating covering only portions of the drape.

FIG. 5 is a schematic depiction of two rolls on a converter depicting a manner for applying a pressure-sensitive adhesive to the dressing.

FIG. 6 a flow diagram depicting an example of a process of assembling the dressing.

### DETAILED DESCRIPTION

The invention is not limited in its application to the details of construction and arrangement of components provided in the following description or illustrated in the attached drawings. The invention is capable of other embodiments and being practiced in various manners. The phrases and terms used herein is for the purpose of description and should not be regarded as limiting. Moreover, the use of “including,” “comprising,” or “having” and variations thereof is meant to encompass the items listed thereafter and equivalents thereof as well as additional items. Directional terms, such as “upper” and “lower,” refer to orientations where a lower portion of an element is positioned closer to patient's skin relative to an upper portion of the same element.

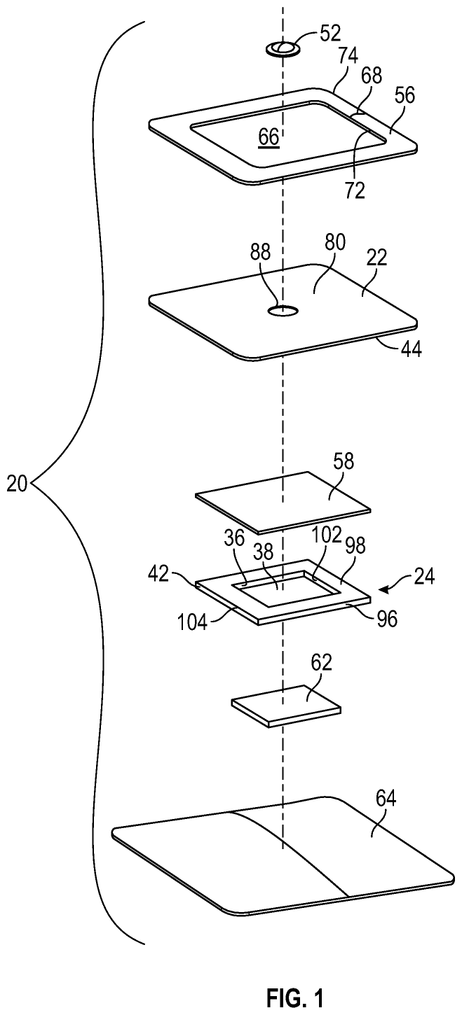

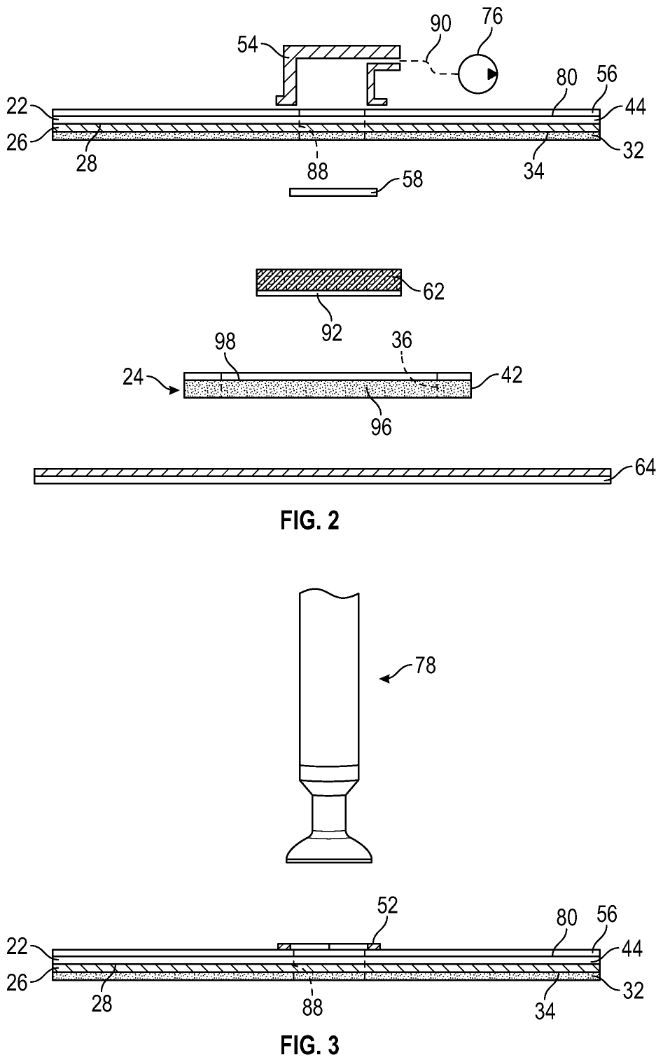

The present disclosure generally relates to negative pressure-type dressings. FIG. 1 depicts an embodiment of a dressing 20 that is useful to provide a pressure that is below normal atmospheric pressure beneath the dressing 20 when the dressing 20 is applied to the patient's skin. The dressing 20 can also be useful when it is desirable to control transmissivity of gases and water vapor through the dressing. The dressing 20 generally includes a drape 22 and a gasket 24. With reference to FIGS. 2 and 3, which are not drawn to scale, a metal coating 26 is provided on a skin-facing surface 28 of the drape 22. A pressure-sensitive adhesive 32 is provided on a lower surface 34 of the metal coating 26, the lower surface 34 being the surface nearest the patient's skin when the dressing 20 is worn. With reference again to FIG. 1, the gasket 24 has a frame shape with an inner edge 36 defining an opening 38 and an outer edge 42 offset inwardly from an outer periphery 44 of the drape 22 leaving a margin of pressure-sensitive adhesive 32 around the gasket 24 when the dressing 20 is finally assembled. The metal coating 26 reduces the transmissivity of oxygen, nitrogen and water vapor through the drape 22, which can be beneficial for negative pressure wound therapy applications as well as other applications.

The dressing 20 can further include a valve 52 (shown in FIG. 1) or a fitting 54 (shown in FIG. 2) depending on the type of vacuum source used with the dressing 20, although neither the valve 52 nor the fitting 54 is required, for example in instances where it is desired only to reduce the transmissivity of oxygen, nitrogen and water vapor through the drape 22 but no negative pressure is to be applied beneath the drape 22. Also optionally, the dressing 20 may also include a casting sheet 56, a filter 58, an absorbent pad 62 and a release liner 64. The arrangement of the components of the dressing 20 and the material from which the components are constructed facilitates conformity of the dressing 20 around a wound, surgical incision, or other tissue site (hereinafter simply referred to as a “tissue site”) so as to maintain a negative pressure environment beneath the drape 22 and around the tissue site, and also allows easier handling for placement of the dressing onto the skin. The dressing 20 can be designed to work with a vacuum source 76, another example of which is shown in FIG. 3, which depicts a spring-loaded mechanical pump 78 described in more detail in U.S. Pat. No. 10,828,202 B1. The dressing 20, however, can be designed to work with other types of pressure sources, e.g., “wall suction” found in hospital rooms as well as a reactor similar to the manner described in U.S. Pat. No. 11,007,083 B2.

The drape 22 is a very thin film-like component capable of maintaining a negative pressure underneath the drape 22 upon application of a vacuum. The drape 22 can be manufactured from a thin film, e.g., less than 2.0 mil (0.050 mm) thickness and preferably about 1.0 mil (0.025 mm) to about 0.8 mil (0.020 mm) thickness, constructed of polyurethane or other semi-permeable material similar to that sold under the Tegaderm® brand or 9834 TPU tape available from 3M. Similar films are also available from other manufacturers. The Tegaderm® brand film, 9834 TPU tape and similar films have a water vapor transmission rate of about 836 g/m²/day, and are appropriate for use when there is a vacuum source continually drawing a vacuum whether it be a mechanical vacuum pump or a solid-state reactor that reacts with a gas in air, e.g., oxygen, to remove the gas from beneath the drape 22.

The thin film from which the drape 22 is made is cast onto the casting sheet 56, which in the illustrated embodiment is made from a polymer such as polyethylene (PE). During assembly of the dressing 20, the casting sheet 56 is kiss cut to provide a casting sheet opening 66. A cross cut 68 can extend from an internal edge 72 of the casting sheet 56, after the material that defines the casting sheet opening 66 has been removed, to an external edge 74 of the casting sheet 56. The external edge 74 of the casting sheet 56 can be coincident with the outer periphery 44 of the drape 22. Because of the inherent flimsiness of the drape 22, by providing a frame-like structure that is the casting sheet 56 connected with the drape 22, application of the dressing 20 is facilitated by way of providing a relatively stiffer or more rigid casting sheet 56 to grasp while placing the dressing 20 over the tissue site. After the dressing 20 has been placed, the casting sheet 56 can be grasped at the cross cut 68 and pulled towards a corner to remove the casting sheet 56 from the drape 22. As is evident in the embodiment depicted in FIG. 1, the casting sheet 56 can be kiss cut around the area of the gasket 24.

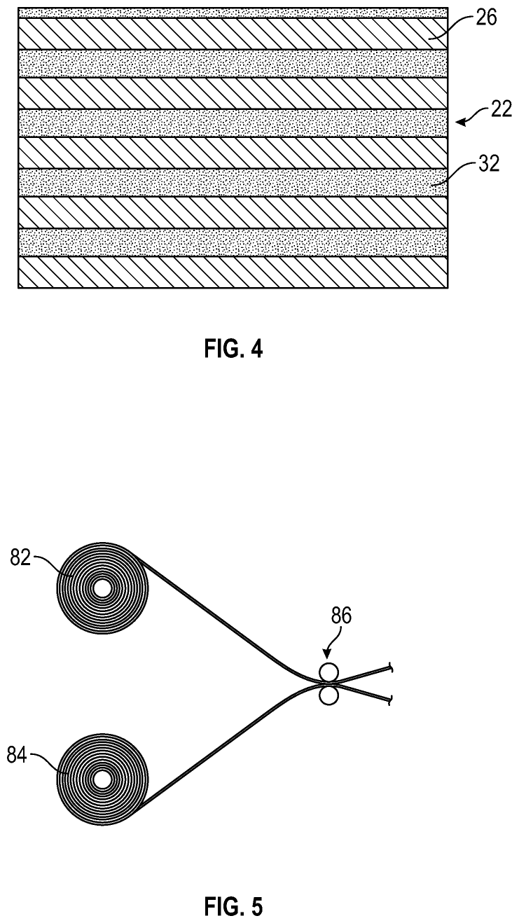

With reference to FIG. 2, the metal coating 26 is provided on the skin-facing surface 28 of the drape 22. With the metal coating 26 applied to the drape 22, it substantially reduces the transmissivity of the drape 22, i.e., the degree to which oxygen, nitrogen and water vapor can pass through the drape 22, in comparison of only providing the drape 22 made up of the thin film for the dressing 20 without the metal coating. The thin film from which the drape 22 is made can be metallized to provide a thin metal layer, which makes up the metal coating 26, on the skin-facing surface 28, which is opposite to an outer surface 80 facing the casting sheet 56. The metal coating 26 can be less than 4.0×10⁻⁵ mm thick so that the total thickness of the drape 22 with the metal coating 26 applied thereto can be less than about 1.0 mil (0.025 mm). Such a metal coating 26 thickness has been found to substantially reduce the transmissivity of oxygen, nitrogen and water vapor through the drape 22 with the metal coating 26 applied thereto, which can be beneficial to maintain negative pressure beneath the drape 22. The metal coating 26 can be less than 2.0×10⁻⁵ mm thick, for example in situations where the transmissivity of oxygen, nitrogen and water vapor through the drape 22 with the metal coating 26 applied thereto is desired to be not as low, e.g., some desired amount of oxygen, nitrogen and water vapor through the drape 22 is desired to allow to be passing through the drape 22. The desired thickness of the metal coating 26 can be adjusted based on the desired transmissivity for the drape 22 with the metal coating applied thereto. The metal coating 26 can be made up of aluminum or other metals approved to be in close contact with human skin. The metal coating 26 can be applied over an entirety of the skin-facing surface 28 of the drape 22. Alternatively, the metal coating 26 can be applied over portions of the skin-facing surface 28 of the drape 22, which is shown in FIG. 4. The metal coating 26 can be applied to the drape 22 using a physical vapor deposition process. Typical paper used for a casting sheet cannot be used in the physical vapor deposition process, hence the use of the polymer casting sheet 56. Masking can be used to allow for the metal coating 26 to be applied over only portions of the skin-facing surface 28 of the drape 22 as opposed to the entirety of the skin-facing surface 28 of the drape 22.

The pressure-sensitive adhesive 32, e.g. less than or about 1.0 mil (0.025 mm) hypoallergenic pressure-sensitive acrylic-based adhesive, is deposited on the metal coating 26 to provide an adhesive layer on the lower surface 34 of the metal coating 26, the lower surface 34 being the surface nearest the patient's skin when the dressing 20 is worn. The pressure-sensitive adhesive 32 can be applied over an entirety of the lower surface 34 of the metal coating 26, which can also be useful during assembly of the dressing 20. When only portions of the skin-facing surface 28 of the drape 22 have the metal coating 26 applied thereon, the pressure-sensitive adhesive 32 is applied to the skin-facing surface 28 of the drape 22.

The casting sheet 56 and the drape 22 with the metal coating 26 deposited thereon can be provided as a pre-manufactured roll 82 (see FIG. 5) of material that is loaded onto a converter (only a portion of which is depicted in FIG. 5). The pressure-sensitive adhesive 32 can be deposited on a paper (or other material) carrier, which is provided on a separate roll, which will be referred to as the adhesive-carrying roll 84. The pre-manufactured roll 82 of the casting sheet 56 and the drape 22 with the metal coating 26 and the adhesive-carrying roll 84 are passed through a nip roller 86. With both rolls 82, 84 traveling through the nip roller 86, the pressure-sensitive adhesive 32 on the paper (or other material) carrier transfers to lower surface 34 of the metal coating 26 (or the skin-facing surface 28 of the drape 22 when the skin-facing surface 28 includes portions not covered by the metal coating 26). After the pressure-sensitive adhesive 32 has been applied, other components such as the valve 52 or fitting 54, the filter 58, the gasket 24, the absorbent pad 62 and the release liner 64 can then be assembled onto the drape 22 by being brought into contact with the pressure-sensitive adhesive 32 to provide the dressing 20.

The drape 22 can also include an aperture 88, which can allow for the connection of the vacuum source(s) 76, 78 as well as other types of vacuum sources, to the dressing 20. As mentioned above, however, the drape 22 need not include an aperture 88, for example in instances where the dressing 20 is not used for negative pressure wound therapy. The aperture 88 can be cut through the casting sheet 56 (prior to removal of the portion of the casting sheet 56 which forms the casting sheet opening 66), the drape 22 and the metal coating 26 within an area surrounded by the gasket 24. In the embodiment depicted in FIG. 1, the valve 52 is placed over or through the aperture 88 to selectively close off the aperture 88 from ambient. In the embodiment depicted in FIG. 2, the fitting 54 is placed over or through the aperture 88 and the fitting 54 can connect to the vacuum source 76 via a hose 90 (depicted schematically).

The absorbent pad 62 is applied onto the pressure-sensitive adhesive 32 on the lower surface 34 of the metal coating 26 and is affixed to the drape 22 via the pressure-sensitive adhesive 32. As more clearly seen in FIG. 1, the absorbent pad 62 is surrounded by the gasket 24. The absorbent material from which the absorbent pad 62 is made can be a super absorbent polyester. Examples of such absorbent materials include a hydroactive wound pad available under the trademark Vilmed®. If desired, a silicone coating 92 (or other skin interface layer) can be provided on a skin-contacting side of the absorbent pad 62.

The gasket 24 can be made up of silicone gel 96 applied onto a backing film 98. The backing film 98 can be a polyurethane, polyethylene, polypropylene, or co-polyester film, that is brought in contact with the pressure-sensitive adhesive 32 on the lower surface 34 of the metal coating 26 to fix the gasket 24 to the drape 22. Typically, silicone does not bond well to an acrylic-based adhesive. By providing the silicone gel 96 on the backing film 98, the gasket 24 can be fixed with respect to the drape 22 while still being able to utilize a pressure-sensitive acrylic-based adhesive on the drape 22 with the metal coating 26. This allows for the benefit of providing a silicone gel 96 with the dressing 20 that can contact the skin around the tissue site and provide a much better seal than only having the pressure-sensitive adhesive, which allows for negative pressure to be obtained underneath the drape 22 around the wound site. The silicone gel 96 can operate as a sealing gasket for the dressing 20 to maintain negative pressure (with respect to atmosphere) underneath the dressing 20. Of the available biomedical materials, silicone gel is identified as a gasket candidate, such as the gel available from Polymer Science, Inc. as part number PS-1050. Other materials, such as hydrogel, could function as the gasket 24 but are not as biocompatible a silicone gel.

With reference to FIG. 1, the backing film 98 has a frame shape with an inner border 102 defining a portion of the opening 38 so that the backing film 98 and the silicone gel 96 surrounds the absorbent pad 62. The backing film 98 has an outer border 104 offset inwardly from the outer periphery 44 the drape 22 so that when the backing film 98 is affixed to the drape 22 with the metal coating 26 a margin of is pressure-sensitive adhesive 32 is positioned around the backing film 98 for affixing to the patient's skin. The silicone gel 96 is positioned on the backing film 98 and can at least substantially match the frame shape of the backing film 98 so as to surround the absorbent pad 62 while leaving the margin of pressure-sensitive adhesive around the gasket 24. This allows the margin of pressure-sensitive adhesive 32 around the gasket 24 to affix to the skin around the tissue site to hold the dressing 20 in place. Since pressure-sensitive adhesive 32 is not particularly suitable for an airtight or watertight seal, the gasket 24 provides an adequate seal so that negative pressure can be maintained within a space beneath the drape 22 and surrounded by the gasket 24.

The filter 58 is an air-permeable/liquid-impermeable filter for covering the aperture 88 in the drape 22. As illustrated in FIG. 1, when the dressing 20 is assembled the air-permeable/liquid-impermeable filter 58 is positioned against the lower surface 34 of the metal coating 26; however, the air-permeable/liquid-impermeable filter 58 can be provided on the outer surface 80 of the drape 22. The air-permeable/liquid-impermeable filter 58 is made from a hydrophobic material such as expanded polytetrafluoroethylene (ePTFE). PTFE is known as being a difficult material to bond because it is “non-stick” by nature. Accordingly, other hydrophobic materials such as microporous membranes available from NXTNano LLC of Claremore, Oklahoma, USA can be used for the air-permeable/liquid-impermeable filter 58. NXTNano LLC provides a microporous membrane constructed of nanofibers having a direct venting of 0.4 cfm while maintaining 14,000 mm hydrostatic head. The NXTNano LLC material is a thermoplastic polyurethane, which makes it easier to affix to the metal coating 26 via the pressure-sensitive adhesive 32 on the skin-facing surface of the lower surface 34 of the metal coating 26.

The release liner 64 contacts the lower surface 34 of the metal coating 26 along the margin of pressure-sensitive adhesive along with the silicone gel 96 (or other gasket material). The release liner 64 also contacts the absorbent pad 62. As such, the release liner 64 should work well with both an acrylic-based adhesive and a silicone gel. In the illustrated example, the release liner 64 is coated with a fluoropolymer release coating on the side of the release liner 64 that contacts the pressure-sensitive adhesive 32 on the lower surface 34 of the metal coating 26 and the appropriate surfaces of the silicone gel 96 and the absorbent pad 62. The release liner 64 can be a polyester film coated on one side with the fluoropolymer release coating, which can be used with silicone adhesives. The release liner 64 has a larger area than the drape 22 and is removed from the lower surface 34 of the metal coating 26 prior to the drape 22 being affixed to a patient's skin around the wound site.

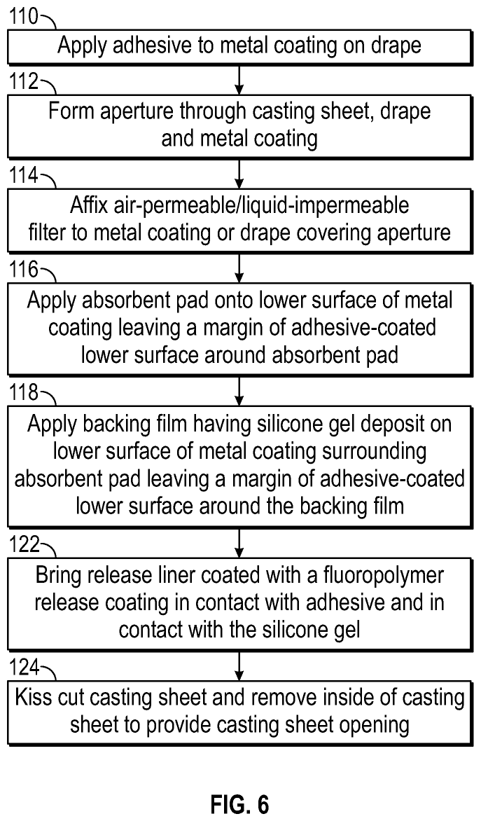

A method of assembling a dressing will be described with reference to the flow diagram shown in FIG. 6 and the embodiment of the dressing shown in FIGS. 1 and 2. The method of assembling the dressing, however, is not limited to only the embodiment of the dressing 20 shown in FIGS. 1 and 2, nor is it limited to the particular order in which the steps are described or shown in FIG. 6, unless otherwise specifically mentioned.

At 110, the pressure-sensitive adhesive 32 can be applied to the lower surface 34 of the metal coating 26. For example, with reference back to FIG. 5 the casting sheet 56 and the drape 22 with the metal coating 26 applied on the skin-facing surface 28 of the drape 22 can be provided as a pre-manufactured roll 82 that is provided on a converter. As discussed above, the pressure-sensitive adhesive 32 can be deposited on a paper (or other material) carrier, which is provided on a separate, adhesive-carrying roll 84. The pre-manufactured roll 82 of the casting sheet 56 and the drape 22 with the metal coating 26 and the adhesive-carrying roll 84 are passed through the nip roller 86 where the pressure-sensitive adhesive 32 on the paper (or other material) carrier transfers to lower surface 34 of the metal coating 26.

With reference back to FIG. 6, at 112, the aperture 88 can be formed through the casting sheet 56, the drape 22 and the metal coating 26. The aperture 88 can be punched or cut through the casting sheet 56, the drape 22 and the metal coating 26, or provided in another manner. The aperture 88 is provided in instances where the dressing 20 is to be used for negative pressure wound therapy.

At 114, when the filter 58 is used with the dressing 20, the filter 58 can be affixed to the drape 22 and the metal coating 26 covering the aperture 88. The filter 58 can be affixed to the lower surface 34 of the metal coating 26 (or the skin-facing surface 28 of the drape 22) via the pressure-sensitive adhesive 32. Alternatively, the filter 58 can be affixed to the outer surface 80 of the drape 22.

At 116, when the absorbent pad 62 is used with the dressing 20, the absorbent pad 62 is applied onto the lower surface 34 of the metal coating 26 (or the skin-facing surface 28 of the drape 22) via the pressure-sensitive adhesive 32. As such, the absorbent pad 62 sticks to the pressure-sensitive adhesive 32 leaving a margin of adhesive-coated lower surface 34 around the absorbent pad 62.

At 118, the backing film 98 having the silicone gel 96 deposited thereon is applied onto the lower surface 34 of the metal coating 26 via the pressure-sensitive adhesive 32 to surround the absorbent pad 62 leaving the margin of adhesive-coated lower surface 34 around the backing film 98.

At 122, the release liner 64 coated with the fluoropolymer release coating is brought in contact with the pressure-sensitive adhesive 32 and also in contact with the silicone gel 96. The fluoropolymer release coating is specially formulated so as to release from both the pressure-sensitive adhesive 32, which can be acrylic based, and the silicone gel 96.

At 124, the casting sheet 56 is kiss cut around the absorbent pad 62 (if provided) and, optionally, the gasket 24 to provide the casting sheet opening 66. An inside portion of the casting sheet 56 is removed, which forms the casting sheet opening 66. The drape 22 is manufactured from a thin film, which allows the operator placing the dressing 20 on the tissue site the ability to identify the absorbent pad 62 (if provided) and the gasket 24 based on the contour of the drape 22 while placing the dressing 20.

A dressing and a process for manufacturing a dressing have been described above with particularity. Modifications and alterations will occur to those upon reading and understanding the preceding detailed description. The invention, however, is not limited to only the embodiments described above. Instead, the invention is broadly defined by the appended claims and the equivalents thereof. It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A dressing comprising:
a drape that is a thin film;
a metal coating provided on a skin-facing surface of the drape;
a pressure-sensitive adhesive provided on a lower surface of the metal coating; and
a gasket having a frame shape with an inner edge defining an opening and an outer edge offset inwardly from an outer periphery of the drape, the gasket being in contact with the pressure-sensitive adhesive provided on the lower surface of the metal coating and leaving a margin of pressure-sensitive adhesive around the gasket.

2. The dressing of claim 1, wherein the drape has a thickness less than 0.05 mm and the metal coating has a thickness of less than 4.0×10⁻⁵ mm.

3. The dressing of claim 1, further comprising a casting sheet, which is a polymer, and the drape is cast onto the casting sheet such that the casting sheet is provided on an outer surface of the drape, which is opposite to the skin-facing surface when the dressing is assembled.

4. The dressing of claim 3, wherein the casting sheet has a frame shape with an internal edge defining a casting sheet opening and an external edge.

5. The dressing of claim 1, wherein the drape includes an aperture extending through the drape and the metal coating, the aperture being surrounded by the gasket.

6. The dressing of claim 5, further comprising a hose fitting or a valve mounted to the drape and in fluid communication with the aperture.

7. The dressing of claim 5, in combination with a vacuum source, wherein the vacuum source is in fluid communication with the aperture.

8. The combination of claim 7, wherein the vacuum source is a reactor configured to consume at least one gas found in air.

9. The dressing of claim 1, further comprising an absorbent pad affixed to the metal coating and the drape via the pressure-sensitive adhesive, the absorbent pad being surrounded by the gasket.

10. The dressing of claim 1, wherein the gasket includes:
a backing film having a frame shape with an inner border defining a portion of the opening and an outer border offset inwardly from the outer periphery of the drape, the backing film being affixed to the metal coating and the drape via the pressure-sensitive adhesive leaving the margin of pressure-sensitive adhesive around the backing film; and
a silicone gel provided on the backing film surrounding the opening.

11. The dressing of claim 1, wherein the metal coating has a thickness of less than 2.0×10⁻⁵ mm.

12. A method of assembling a dressing comprising:
providing as a pre-manufactured roll a casting sheet, a drape and a metal coating provided on a skin-facing surface of the drape;
coating a pressure-sensitive adhesive on the metal coating; and
applying a backing film having a silicone gel deposited thereon onto the pressure-sensitive adhesive, wherein the backing film has frame shape with an inner border defining an opening and an outer border offset from an outer periphery of the drape leaving a margin of pressure-sensitive adhesive around the backing film, and the silicone gel surrounds the opening.

13. The method of claim 12, further comprising:
forming an aperture through the casting sheet, the drape and the metal coating.

14. The method of claim 13, further comprising:
kiss cutting the casting sheet around the aperture; and
removing an inside portion of casting sheet.

15. The method of claim 12, further comprising:
applying an absorbent pad onto the pressure-sensitive adhesive.

16. The method of claim 15, further comprising:
kiss cutting the casting sheet around the absorbent pad; and
removing an inside portion of casting sheet.

17. The method of claim 14, further comprising:
affixing a hose fitting or a valve to the drape that is in fluid communication with the aperture.

18. The method of claim 12, wherein providing as the pre-manufactured roll the casting sheet, the drape and the metal coating provided on the skin-facing surface of the drape further includes the metal coating being applied over only portions of the skin-facing surface of the drape as opposed to the entirety of the skin-facing surface of the drape.

19. The dressing of claim 1, wherein the metal coating provided over portions of the skin-facing surface of the drape as opposed to the entirety of the skin-facing surface of the drape.

20. The dressing of claim 19, wherein the pressure-sensitive adhesive is applied to the skin-facing surface of the drape

21. A roll of material for manufacturing a dressing, the roll comprising:
a casting sheet, which is a polymer;
a film, which is less than 2.0 mil (0.050 mm) in thickness, cast onto the casting sheet; and
a metal coating, which is less than 4.0×10⁻⁵, applied to the film on a surface of the film that is opposite the casting sheet.

22. The roll of claim 21, wherein the film is less than 1.0 mil (0.025 mm) in thickness.

23. The roll of claim 21, wherein the metal coating is less than 4.0×10⁻⁵ in thickness.

24. The roll of claim 21, wherein the metal coating includes aluminum.

25. The roll of claim 21, wherein the metal coating is applied over an entirety of the surface of the film that is opposite the casting sheet.

27. The roll of claim 21, wherein the metal coating is applied over a portion of the surface of the film that is opposite the casting sheet.
